(19) United States
(12) Patent Application Publication
(10) Pub. No.: US 2017/0007469 A1
(43) Pub. Date: Jan. 12, 2017

(54) Feminine Hygiene System

(71) Applicant: Michelle Medley, Middletown, NY (US)

(72) Inventor: Michelle Medley, Middletown, NY (US)

(21) Appl. No.: 14/796,273

(22) Filed: Jul. 10, 2015

(51) Int. Cl. A61F 13/472; A61F 13/496

(52) U.S. Cl. CPC A61F 13/472; A61F 13/496

(57) ABSTRACT

A feminine hygiene system for retaining a sanitary napkin on a user's genitalia includes an undergarment that may be worn by a user such that the undergarment covers the user's genitalia. The undergarment comprises an inner layer and an outer layer. A sanitary napkin is selectively positioned on the undergarment. The sanitary napkin is positioned on the inner layer such that the sanitary napkin may absorb menstrual fluids when the undergarment is worn by the user.

### BACKGROUND OF THE DISCLOSURE

### Field of the Disclosure

The disclosure relates to hygiene devices and more particularly pertains to a new hygiene device for retaining a sanitary napkin on a user's genitalia.

### SUMMARY OF THE DISCLOSURE

An embodiment of the disclosure meets the needs presented above by generally comprising an undergarment that may be worn by a user such that the undergarment covers the user's genitalia. The undergarment comprises an inner layer and an outer layer. A sanitary napkin is selectively positioned on the undergarment. The sanitary napkin is positioned on the inner layer such that the sanitary napkin may absorb menstrual fluids when the undergarment is worn by the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a feminine hygiene system according to an embodiment of the disclosure.

FIG. 2 is a back view of an embodiment of the disclosure.

FIG. 3 is a front view of an embodiment of the disclosure.

FIG. 4 is a perspective in-use view of an embodiment of the disclosure.

### DESCRIPTION OF THE PREFERRED EMBODIMENT

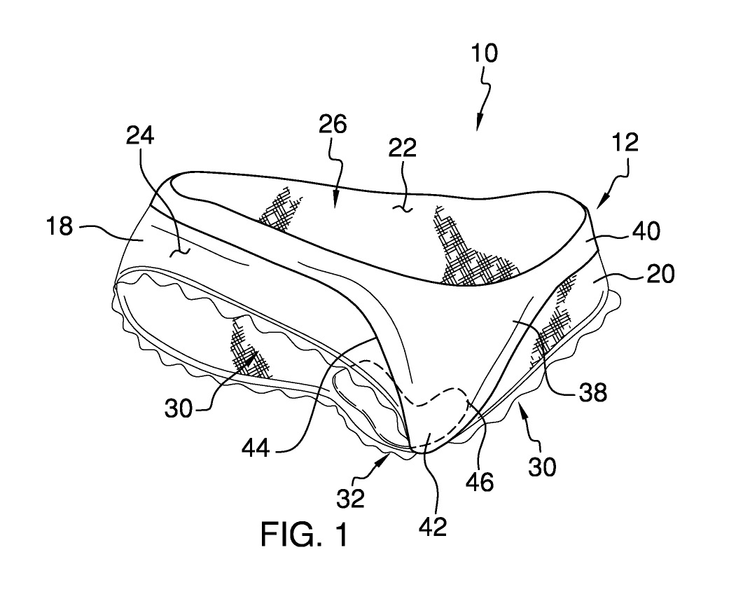

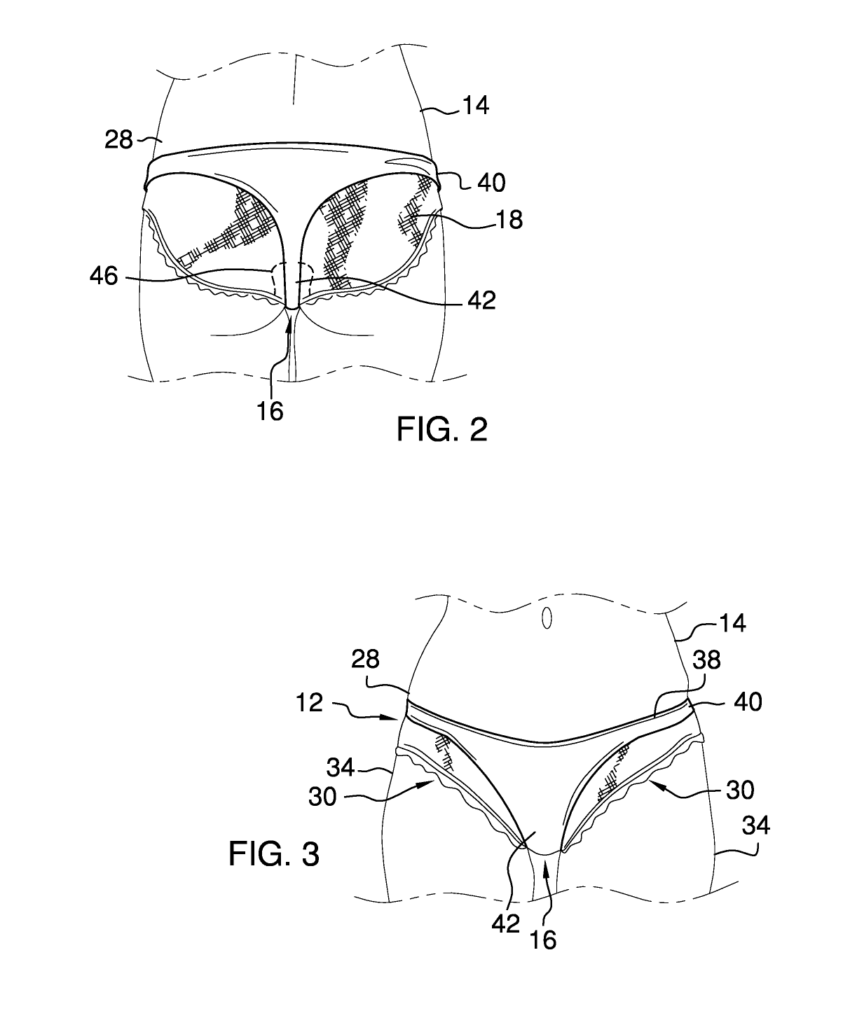

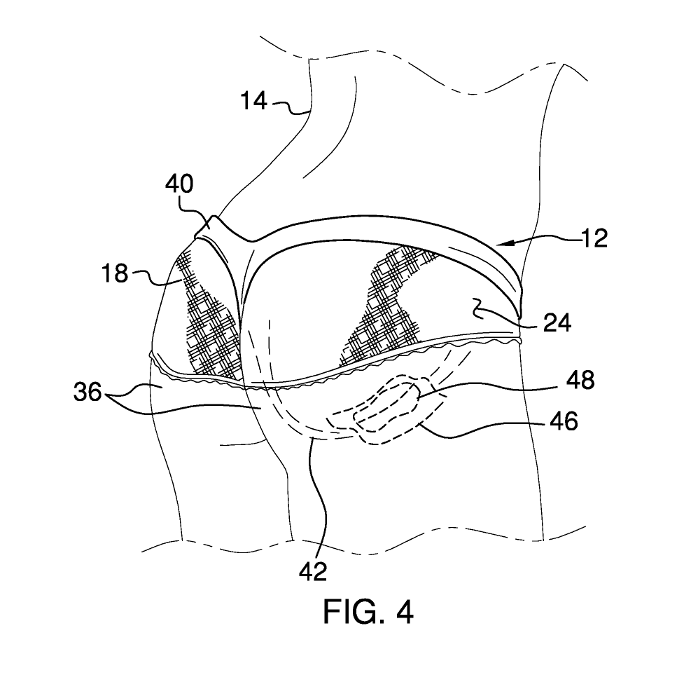

With reference now to the drawings, and in particular to FIGS. 1 through 4 thereof, a new hygiene device embodying the principles and concepts of an embodiment of the disclosure and generally designated by the reference numeral 10 will be described.

As best illustrated in FIGS. 1 through 4, the feminine hygiene system 10 generally comprises an undergarment 12 that may be worn by a user 14 such that the undergarment 12 covers the user's genitalia 16. The user 14 may be a menstruating female. The undergarment 12 comprises an inner layer 18 that has an outer wall 20 and the outer wall 20 has an inner surface 22 and an outer surface 24. The inner layer 18 is continuous such that the inner layer 18 forms a closed loop. The inner layer 18 has a waist opening 26 such that the waist opening may be worn around a waist 28 of the user 14. The inner layer 18 has a pair of leg openings 30 that are each separated by a crotch section 32 of the inner layer 18. Thus, each of the leg openings 30 may insertably receive one of a pair of legs 34 of the user 14 having the crotch section covering the user's genitalia 16. The inner layer 18 extends downwardly along each of a pair of buttocks 36 of the user 14 when the user 14 wears the undergarment 12.

The undergarment 12 further includes an outer layer 38 that has a first band 40 and a second band 42. The first band 40 is continuous such that the first band 40 forms a closed loop. The second band 42 is coupled to the first band 40 such that the second band 42 extends between opposing sides of the first band 40. The outer layer 38 has an inside surface 44 and the inside surface 44 of the outer layer 38 is attached to the outer surface 24 of the inner layer 18. The first band 40 is coextensive with the waist opening 26 and the second band 42 extends along the crotch section 32 of the inner layer 18. The second band 42 extends between each of the user's buttock 36 and across the user's genitalia 16 when the user 14 wears the undergarment 12.

A sanitary napkin 46 is selectively positioned on the undergarment 12 such that the sanitary napkin 46 may absorb menstrual fluids 48 when the undergarment 12 is worn by the user 14. The sanitary napkin 46 is positioned on the inner surface 22 corresponding to the crotch section 32 such that the sanitary napkin may be positioned over the user's genitalia 16. The second band 42 urges the sanitary napkin 46 against the user's genitalia 16 such that the sanitary napkin 46 may be held in place on the user's genitalia 16 thereby inhibiting the menstrual fluids 48 from escaping the undergarment 12. The sanitary napkin 46 may be a sanitary napkin 46 of any conventional design.

In use, the sanitary napkin 46 is positioned on the inner surface 22 corresponding to the crotch section 32. The undergarment 12 is worn such that the sanitary napkin 46 covers the user's genitalia 16. The second band 42 urges the sanitary napkin 46 against the user's genitalia 16 thereby inhibiting the sanitary napkin 46 from moving while the user 14 wears the undergarment 12. Thus, the undergarment 12 reduces the possibility of sanitary napkin 46 from being displaced from the user's genitalia 16 thereby inhibiting the menstrual fluid 48 from escaping the undergarment 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A feminine hygiene system comprising:
an undergarment configured to be worn by a user such that said undergarment covers the user's genitalia, said undergarment comprising an inner layer and an outer layer; and
a sanitary napkin being selectively positioned on said undergarment, said sanitary napkin being positioned on said inner layer wherein said sanitary napkin is configured to absorb menstrual fluids when said undergarment is worn by the user.

2. The assembly according to claim 1, wherein said inner layer has an outer wall, said outer wall having an inner surface and an outer surface, said inner layer being continuous such that said inner layer forms a closed loop.

3. The assembly according to claim 1, wherein said inner layer has a waist opening wherein said waist opening is configured to be worn around a waist of the user, said inner layer having a pair of leg openings each being separated by a crotch section of said inner layer wherein each of said leg openings is configured to insertably receive one of a pair of legs of the user having said crotch section covering the user's genitalia.

4. The assembly according to claim 1, wherein said outer layer has a first band and a second band, said first band being continuous such that said first band forms a closed loop, said second band being coupled to said first band such that said second band extends between opposing sides of said first band.

5. The assembly according to claim 4, wherein:
said inner layer has an outer surface, a waist opening and a crotch section; and
said outer layer has an inside surface, said inside surface of said outer layer being attached to said outer surface of said inner layer, said first band being coextensive with said waist opening, said second band extending along said crotch section of said inner layer.

6. The assembly according to claim 1, wherein:
said outer layer has an inner surface and a crotch section; and
said sanitary napkin is positioned on said inner surface corresponding to said crotch section wherein said sanitary napkin is configured to be positioned over the user's genitalia.

7. The assembly according to claim 6, wherein said second band urges said sanitary napkin against the user's genitalia wherein said sanitary napkin is configured to be held in place on the user's genitalia thereby inhibiting the menstrual fluids from escaping said undergarment.

8. A feminine hygiene system comprising:
an undergarment configured to be worn by a user such that said undergarment covers the user's genitalia, said undergarment comprising:
an inner layer having an outer wall, said outer wall having an inner surface and an outer surface, said inner layer being continuous such that said inner layer forms a closed loop, said inner layer having a waist opening wherein said waist opening is configured to be worn around a waist of the user, said inner layer having a pair of leg openings each being separated by a crotch section of said inner layer wherein each of said leg openings is configured to insertably receive one of a pair of legs of the user having said crotch section covering the user's genitalia, and
an outer layer having a first band and a second band, said first band being continuous such that said first band forms a closed loop, said second band being coupled to said first band such that said second band extends between opposing sides of said first band, said outer layer having an inside surface, said inside surface of said outer layer being attached to said outer surface of said inner layer, said first band being coextensive with said waist opening, said second band extending along said crotch section of said inner layer; and
a sanitary napkin being selectively positioned on said undergarment wherein said sanitary napkin is configured to absorb menstrual fluids when said undergarment is worn by the user, said sanitary napkin being positioned on said inner surface corresponding to said crotch section wherein said sanitary napkin is configured to be positioned over the user's genitalia, said second band urging said sanitary napkin against the user's genitalia wherein said sanitary napkin is configured to be held in place on the user's genitalia thereby inhibiting the menstrual fluids from escaping said undergarment.
